(19) United States
(12) Patent Application Publication
(10) Pub. No.: US 2011/0202057 A1
(43) Pub. Date: Aug. 18, 2011

(54) ELECTROSURGICAL TOOL WITH MOVEABLE ELECTRODE THAT CAN BE OPERATED IN A CUTTING MODE OR A COAGULATION MODE

(76) Inventors: Jonathan O. Thorne, Boulder, CO (US); Kevin Morningstar, Louisville, CO (US)

(21) Appl. No.: 13/096,568

(22) Filed: Apr. 28, 2011

Related U.S. Application Data
(63) Continuation of application No. 12/350,078, filed on Jan. 7, 2009, now abandoned.
Application No. 12/350,078 is a division of application No. 11/146,867, filed on Jun. 7, 2005, now abandoned.
(60) Provisional application No. 60/578,138, filed on Jun. 8, 2004.

(51) Int. Cl. A61B 18/14

(52) U.S. Cl. 606/48

(57) ABSTRACT

An electrosurgical instrument is provided. The electrosurgical instrument includes an active electrode in close proximity to a return electrode. The active electrode has a first thermal diffusivity. The second electrode has a second thermal diffusivity greater than the first thermal diffusivity. The volume, shape, and thermal diffusivity of the second electrode facilitate the transport of heat.

### CROSS-REFERENCE TO RELATED APPLICATIONS

The present application claims the benefit of U.S. Utility patent application Ser. No. 12/350,078, titled ELECTROSURGICAL TOOL WITH MOVEABLE ELECTRODE THAT CAN BE OPERATED IN A CUTTING MODE OR A COAGULATION MODE, filed 7 Jan. 2009. The '078 application claims the benefit of U.S. Utility patent application Ser. No. 11/146,867, titled ELECTROSURGICAL CUTTING INSTRUMENT, filed 7 Jun. 2005, which claims the benefit of U.S. Provisional Patent Application No. 60/578,138, titled BIPOLAR ELECTROSURGICAL CUTTING INSTRUMENT, filed 8 Jun. 2004, all of which are incorporated herein as in by reference.

### FIELD OF THE INVENTION

The present invention relates to electrosurgical instruments and, more particularly, to a bipolar electrosurgical instrument useful to cut tissue.

### BACKGROUND OF THE INVENTION

Doctors and surgeons have used electrosurgery for many decades. In use, electrosurgery consists of applying electrical energy to tissue using an active and a return electrode. Typically, a specially designed electrosurgical generator provides alternating current at radio frequency to the electrosurgical instrument, which in turn contacts tissue. Other power sources are, of course, possible. The art of design and production of electrosurgical generators is well known.

Electrosurgery includes both monopolar electrosurgery and bipolar electrosurgery. Monopolar electrosurgery is somewhat of a misnomer as the surgery uses two electrodes. A surgeon handles a single, active electrode while the second electrode is usually grounded to the patient at a large tissue mass, such as, for example, the gluteus. The second electrode is typically large and attached to a large tissue mass to dissipate the electrical energy without harming the patient. Bipolar electrosurgical instruments differ from monopolar electrosurgical instruments in that the instrument itself contains both the active and return electrode.

In monopolar electrosurgery, or monopolar surgery, or monopolar mode, the patient is grounded using a large return electrode, also referred to as a dispersive electrode or grounding pad. This return electrode is typically at least six (6) square inches in area. The return electrode is attached to the patient and connected electrically to the electrosurgical generator. Most return electrodes today employ an adhesive to attach the electrode to the patient. Typically the return electrode is attached on or around the buttocks region of the patient. A surgical electrode (active electrode) is then connected to the generator. The generator produces the radio frequency energy and when the active electrode comes in contact with the patient the circuit is completed. Certain physiological effects occur at the active electrode-tissue interface depending on generator power levels and waveform output, active electrode size and shape, as well as tissue composition and other factors. These effects include tissue cutting, coagulation of bleeding vessels, ablation of tissue and tissue sealing.

While functional, monopolar surgery has several drawbacks and dangers. One problem is that electrical current needs to flow through the patient between the active electrode and the ground pad. Because the electrical resistance of the patient is relatively high, the power levels used to get the desired effects to the tissue are typically high. Nerve and vessel damage is not uncommon. Another problem includes unintended patient burns. The burns occur from, among other things, current leakage near the active or return electrode and touching of other metal surgical instruments with the active electrode. Another problem is capacitive coupling of metal instruments near the active electrode causing burns or cauterization in unintended areas. Yet another problem includes electrical burns around the ground or return pad because electrical contact between the patient and the ground pad deteriorates at one or more locations. These and other problems make monopolar electrosurgical instruments less than satisfactory.

The drawbacks and problems associated with monopolar surgery resulted in the emergence of bipolar electrosurgery in the mid-twentieth century. With bipolar electrosurgery, the active and ground electrode are proximal to one another, and typically on the same tool. The ground being on the instrument allowed for the removal of the grounding pad and the problems associated therein. Moreover, because the electrical energy only flows between the instrument electrodes, the current flows through the patient only a short distance, thus the resistance and the power required are both lower. This substantially reduces the risk of nerve or vessel damage or unintentional patient burns. Bipolar surgery works very well for coagulation, ablation and vessel sealing.

While bipolar instruments solved many problems associated with monopolar instruments, attempts at creating a bipolar cutting instrument that resembles a monopolar cutting instrument have been largely unsuccessful. In order to have smooth cutting, the energy density and heat generated proximal to the cutting electrode must be great enough to cause the adjacent tissue cells to explode. This thin line of exploding cells is what causes tissue to part when cutting occurs. If the power density and heat are not high enough, the cells fluid will slowly boil off and tissue desiccation and coagulation will occur. Attempts to make a bipolar instrument with two electrodes or blades proximal to each other have not resulted in the desired smooth cutting effect, mostly because a high enough current density could not be achieved and one or both of the electrodes started to stick to the tissue.

U.S. Pat. No. 4,202,337 (Hren et al.) describes an electrosurgical instrument similar to a blade with side return electrodes with an active area that is 0.7 to 2.0 times the active electrode area. This invention does not recognize the need to quickly dissipate the heat from the surface of the return electrode, that is the heat generated at the tissue-electrode interface. It also does not recognize a need to transport the heat away from return electrode. Indeed, the inventor states that the return electrodes should be a thin metalized substance such as silver which is silk screen applied to the ceramic and then fired (7-33 through 7-36). Because the thin metalized substance does not have sufficient volume to transport away or store the heat generated during use, the return electrode of this invention will quickly heat up and start to stick and drag making it unsuitable for most surgical applications.

U.S. Pat. No. 5,484,435 (Fleenor et al.) describes a bipolar cutting instrument in which the return electrode, or shoe, that moves out of the way as the instrument is drawn through the tissue. The discussion is that the passive or return electrode should be at least three times the area of the active electrode. This invention also does not recognize the need to quickly dissipate the heat from the surface of the return electrode, that is the heat generated at the tissue-electrode interface and also does not recognize a need to transport the heat away from return electrode. When in use the return electrode of this invention will also quickly heat up and start to stick and drag making it unsuitable for most surgical applications. In addition, the requirement that one electrode spring or move out of the way makes it unusable for many procedures.

It is against this background and the desire to solve the problems of the prior art, that the present invention has been developed.

### SUMMARY OF THE INVENTION

To attain the advantages and in accordance with the purpose of the invention, as embodied and broadly described herein, a electrosurgical device or instrument is provided. The electrosurgical instrument comprises an active electrode and a return electrode residing in close proximity. The active electrode made of a first material with a first thermal diffusivity. The return electrode made of a second material with a second thermal diffusivity greater than the first thermal diffusivity. The volume of the second material, the geometry of the second material, and the thermal diffusivity of the second material being sufficient to facilitate the transport of heat from the surface of the at least one return electrode.

The foregoing and other features, utilities and advantages of the invention will be apparent from the following more particular description of a preferred embodiment of the invention as illustrated in the accompanying drawings.

### BRIEF DESCRIPTION OF THE DRAWINGS

The accompanying drawings, which are incorporated in and constitute a part of this specification, illustrate embodiments of the present invention, and together with the description, serve to explain the principles thereof. Like items in the drawings are referred to using the same numerical reference.

FIG. 1 illustrates a conventional electrosurgical system in functional block diagrams with the invention connected to this system.

FIG. 2 is a view of an electrosurgical instrument consistent with one embodiment of the present invention.

FIG. 3 is a cross sectional view of the electrosurgical instrument tip shown in FIG. 2.

FIG. 4 is a cross sectional view of the electrosurgical instrument tip shown in FIG. 2.

FIG. 5 is a view of another electrosurgical instrument tip consistent with one embodiment of the present invention.

FIG. 6 is a cross sectional view of the electrosurgical instrument tip shown in FIG. 5.

FIG. 7 is a cross sectional view of the electrosurgical instrument tip shown in FIG. 5.

FIG. 8 is a view of another electrosurgical instrument consistent with one embodiment of the present invention.

FIG. 9 shows the electrosurgical instrument tip of FIG. 8 in more detail.

FIG. 10 is a view of another electrosurgical instrument consistent with one embodiment of the present invention.

FIG. 11 is a cross-sectional view the electrosurgical instrument tip of FIG. 10 in an extended position.

FIG. 12 is a cross-sectional view the electrosurgical instrument tip of FIG. 10 in a retracted position.

FIG. 13 is a cross-sectional view the electrosurgical instrument tip e of FIG. 10 in an extended or retracted position.

FIG. 14 is a view of another electrosurgical instrument tip consistent with one embodiment of the present invention.

FIG. 15 is a cross-sectional view of the electrosurgical instrument tip of FIG. 14.

FIG. 16 is a view of an alternate embodiment of the electrosurgical instrument shown in FIG. 14 with a suction cannula attached.

FIG. 17 is a view of another embodiment of the present invention incorporated into a bipolar electrosurgical forceps.

FIG. 18 shows the electrosurgical instrument tip of FIG. 17 in more detail.

FIG. 19 is an end view the cutting tine of the bipolar electrosurgical forceps instrument tip of FIG. 18.

FIG. 20 is a cross-sectional view of the cutting tine of the bipolar electrosurgical forceps instrument tip of FIG. 18.

FIG. 21 is a side view of another embodiment of the present invention incorporating a loop cutting electrode into one tine of a bipolar electrosurgical forceps.

FIG. 22 is a top view of the embodiment of FIG. 21 showing the loop cutting electrode extended.

FIG. 23 is a top view of the embodiment of FIG. 21 showing the loop cutting electrode retracted.

### DETAILED DESCRIPTION

The present invention will now be described with reference to the figures. While embodiments of the invention are described, one of ordinary skill in the art will recognize numerous shapes, sizes, and dimensions for the actual instruments are possible. Thus, the specific embodiments described and shown herein should be considered exemplary and non-limiting.

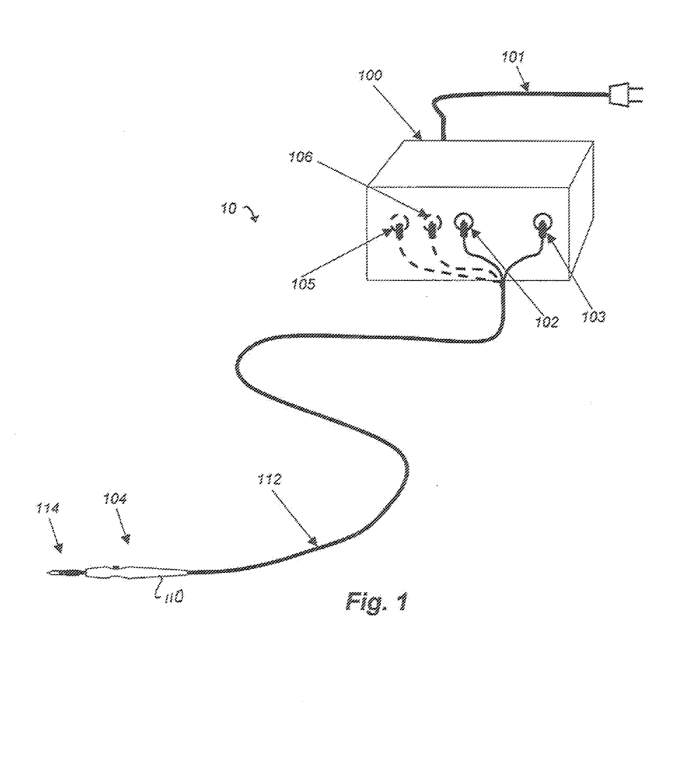

FIG. 1 shows an electrosurgical system 10 consistent with an embodiment of the present invention. System 10 includes a bipolar electrosurgical generator 100. Electrosurgical generator 100 may include its own power source, but is typically powered using standard AC wall current via a power cord 101. Electrosurgical generator 100 uses power, such as, AC wall current to generate a radio frequency output of various waveforms to facilitate cutting, coagulation and other physiological effects to the tissue. Electrosurgical generator 100 and the various radio frequency outputs are well known in the art and not explained further herein. Electrosurgical generator 100 includes connections 102 and 103. Optionally, second connectors 105 and 106 may be provided also as shown in phantom. One connection, such as, for example, connection 102, provides electrical power or is an electrical power source to the instrument while the other connection, such as for example, connection 103 is a ground for the electrical power source. System 10 also includes a device 104 having a handle 110 and a pair of electrodes in an electrosurgical instrument tip 114. The electrosurgical instrument tip 114 is explained further below. Device 104 is connected to connections 102 and 103 of electrosurgical generator 100 using any conventional means, such as, for example, cable 112. Optional connectors 105 and 106 may be used for actuation of the electrosurgical generator, switching between waveforms and instrument identification. The operating principles of these functions are well known in the art.

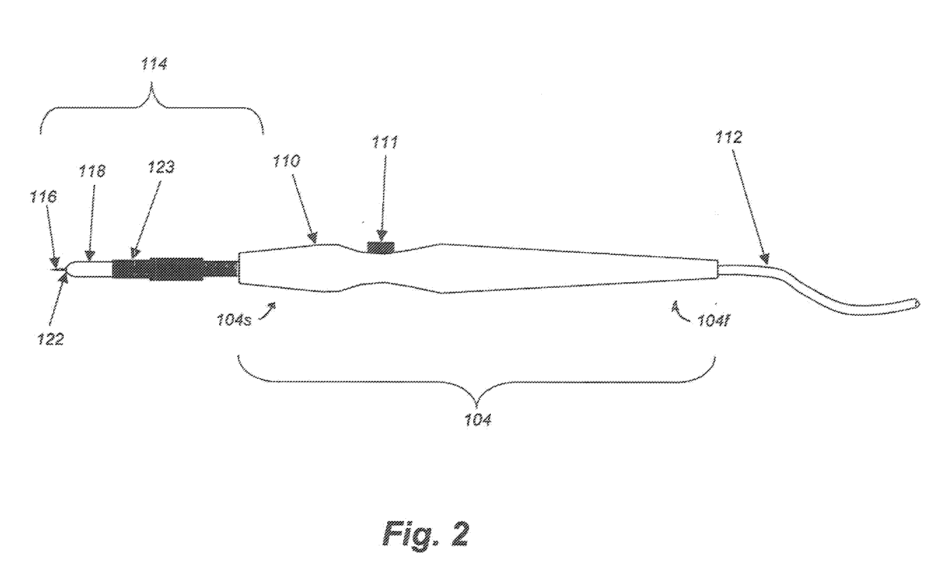

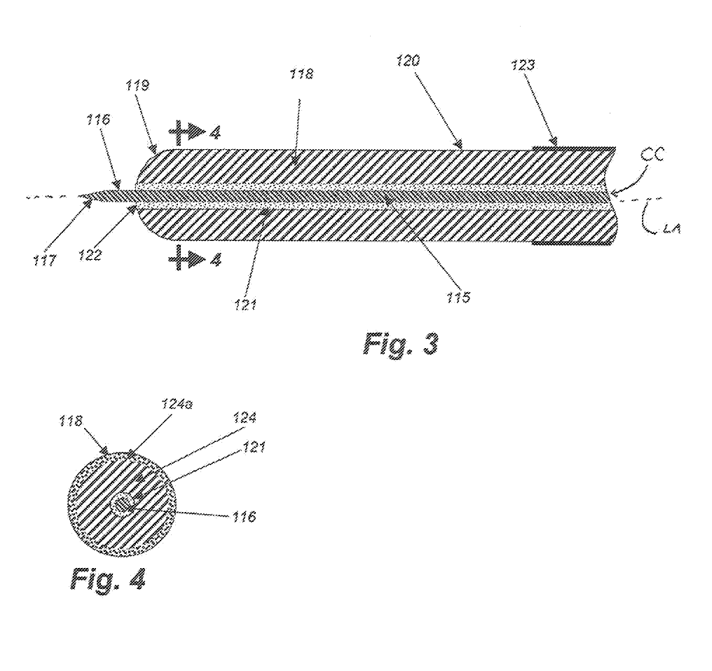

FIG. 2 shows device 104 with the electrosurgical instrument tip 114 in more detail. Power is supplied to device 104 from cable 112. Connecting cable 112 to device 104 is conventionally known. Generally, as shown, cable 112 is arranged at a first end 104f of device 104 and electrodes 114 are arranged at a second end 104s of device 104, but alternative configurations are possible. The electrical power source provides radio frequency energy through cable 112 and a handle 110 of device 104 to the electrodes 116 and 118. The electrosurgical instrument tip 114 includes an active or, in cutting applications, a cutting electrode 115 (see FIG. 3) having an exposed active electrode tip 116 and a return or ground electrode 118. Cable 112 provides a path from connection 102, the electrical power source, to active electrode 115 and a return path to a ground at connection 103 from return electrode 118.

Active electrode 115 and return electrode 118 are separated in close proximity to each other and separated by an insulative material 121 (see FIG. 3), normally a dielectric such as plastic or ceramic. In some cases this insulative material may simply be air or other gases. As shown in FIGS. 2 and 3, active electrode 115 extends along a longitudinal axis LA from a center cavity CC in return electrode 118. Because active electrode 115 and return electrode 118 may short along central cavity CC, insulative material 121 may be provided to inhibit shorting or the like. The portion of active electrode 115 extending beyond return electrode 118 along the longitudinal axis LA is active electrode tip 116 and is separated from return electrode 118 by air. Alternative construction of the electrodes may require more, less, or no insulative material 121. It is believed the material and dimensional properties of the return electrode 118 as related to active electrode tip 116 facilitates operation of the current invention.

Electrodes 116 and 118 are coupled to connector housing 123. Connector housing 123 may be an insulative material and/or wrapped with an insulative material. Connector housing 123 is coupled or plugged into handle 110 in a manner known to those versed in the art of monopolar electrosurgery. Handle 110 may include one or more power actuators 111 to allow the activation of the bipolar generator and give the user the ability to switch between different waveform outputs and power levels. For example, the signals to facilitate this may be supplied through separate connections, such as connectors 105 and/or 106. The operation and configuration of such power actuators to activate the generator are well known to those versed in the field of electrosurgery and are now commonly used in monopolar electrosurgery. Actuators 111 could include buttons, toggle switches, pressure switches, or the like. Connections 102, 103, 105 and 106 can be combined into a single plug at the generator.

Referring to FIG. 3, which is a cross-sectional view of the electrosurgical instrument tip 114 shown in FIGS. 1 and 2, active electrode 115, including active electrode tip 116, may be constructed from a material with a high melting point, such as, for example, tungsten and some stainless steel alloys. Active electrode tip 116 has an area and can be exposed to tissue. Active electrode tip 116 may be shaped into an edge 117, which may be shaped such as, for example, a blade, dowel, wedge, point, hook, elongated, or the like to facilitate use of device 104. Active electrode tip 116 is generally exposed so as to be capable of contacting tissue. The portion of active electrode 115 extending along central cavity CC is covered by electrical insulative material 121, a part of which may extend beyond central cavity CC, such as insulative tip 122. The electrical insulator 121 electrically insulates the active electrode 115 from the return electrode 118. The size of the active area of the electrode 116 is important to the function of the device. For example, if the size of this electrode is too large relative to other characteristics of the return electrode, the device may not function properly.

Referring now to the return electrode 118, to facilitate the transport of heat from the surface, at least the surface of this electrode and/or a portion of some depth into this electrode should be made of a material with a relatively high thermal diffusivity. Dissipation of localized hot spots is a function of the thermal diffusivity (α) of the electrode material. Hot spots occur where sparking or arching occurs between the tissue and the electrode. These hot spots are where sticking of tissue to the electrode occurs. The higher the thermal diffusivity, the faster the propagation of heat is through a medium. If heat is propagated away fast enough, hot spots are dissipated and the sticking of tissue to the electrode does not occur.

The thermal diffusivity of a material is equal to the thermal conductivity (k) divided by the product of the density (ρ) and the specific heat capacity (Cₚ).

Thus

$$α = \frac{k}{ρ \cdot {C}_{p}}$$

In most electrosurgery applications, a thermal diffusivity of at least 1.5×10⁻⁵ m²/s works to reduce tissue sticking to the electrode. An electrode made of or coated with a sufficient thickness, volume and geometry of higher thermal diffusivity material works significantly better to reduce sticking. A lower thermal diffusivity would work for lower power applications. It has been found that high thermal diffusivity, such as materials with a thermal diffusity of 9.0×10⁻⁵ m²/s, works well in the present invention. Materials with these high thermal diffusity rates still need sufficient volume to work. Suitable materials for the return electrode, or at least a portion of the outer surface of the electrode include silver, gold, and alloys thereof. Copper and aluminum may also be used, however a coating of other material must be used in order to achieve biocompatibility. For example, referring to FIG. 3, return electrode 118 is a solid material of biocompatible material. Referring to FIG. 4, however, return electrode 118 may have a core material 124 with a surface coating or plating 124a of a sufficient thickness of high thermal diffusivity material. Tungsten and Nickel are less desirable material for the return electrode, but can be made to work in some embodiments. A table showing thermal properties of electrode materials is shown below.

TABLE I

| | SPECIFIC | | | |
|---|---|---|---|---|
| | HEAT | THERMAL | | |
| | CAPACITY | CON- | | |
| | Cₚ × 10⁻² | DUCTIVITY | DENSITY | THERMAL |
| | Joules/ | kW/ | ρ | DIFFUSIVITY |
| MATERIAL | (Kg · °K) | (m · °K) | kg/m³ | α × 10⁵ m²/s |
| Silver | 2.39 | 415 | 10,500 | 16.6 |
| Gold | 1.30 | 293 | 19,320 | 11.7 |
| Copper | 3.85 | 386 | 8,890 | 10.27 |
| Aluminum | 9.38 | 229 | 2,701 | 9.16 |
| Tungsten | 1.34 | 160 | 19,320 | 6.30 |
| Nickel | 4.56 | 93.0 | 8,910 | 2.24 |
| Stainless | 4.61 | 16.0 | 7,820 | 0.44 |

### Steel

A relatively high thermal diffusivity material at the surface of the return electrode facilitates dissipating the high temperatures that occur at the point of sparking during electrosurgery at the tissue-electrode interface. The temperature of the sparks may exceed 1000° C. If even a tiny area on the surface of the electrode is heated from the energy of the spark and the surface temperature at that point exceeds 90° C., sticking of tissue to that point is likely to occur. If sticking occurs, the instrument will drag and eschar will build up, making the instrument unsuitable for use.

In addition to having a relatively high thermal diffusivity, the return electrode should have thermal mass to assist in heat transport. The thermal mass inhibits the overall electrode from heating up to a temperature where sticking occurs. The geometry of the high diffusivity material of the return electrode should also be designed to facilitate flow of heat away from the surface and distal portion of the return electrode. As shown, the body of the return electrode 118 is provided with a larger cross-sectional area and enough thermal mass such that for most electrosurgery applications the overall electrode will remain below the temperature at which sticking will occur. For higher power electrosurgery applications, where more heat must be dissipated, the length or cross sectional area of the electrode can be increased as one moves distally away from the electrode tip. If a plated or coated return electrode is used, the cross sectional area of the portion of the electrode made of the high thermal diffusivity material should either remain constant or increase when one moves distally away from the return electrode tip. If the cross sectional area of the high thermal diffusivity material diminishes or necks down along the length of the electrode, this will restrict heat flow away from the tip and may diminish the operational performance of the device. Analysis and experimentation has shown that when using a material with a thermal diffusivity greater than 9.0×10⁻⁵ m²/s for the return electrode, and a relatively small active electrode less than 1 cm in length, that the return electrode mass should be at least 0.5 grams to facilitate good cutting. For larger active electrodes, the mass of the return electrode or portion of the return electrode made out of material with a high coefficient of thermal diffusivity should be greater such as, for example, greater than 1.0 grams, and for some geometries, substantially greater. Conversely, for very small active electrodes, the mass of the return electrode can be much less. The shape of the return electrode should also be optimized to facilitate flow of heat away from the electrode surface. When referring to the electrode mass in the above discussion, this is defined as the mass of the portion of the electrode that dissipates the thermal energy during electrosurgery. Thus certain portions of the instrument that are electrically connected to the electrodes, but do not significantly contribute to dissipation of thermal energy, such as a long shaft connected to the tip, may be of significantly higher mass than as outlined in the above discussion. Lastly, materials with higher thermal diffusivity tend to require less thermal mass than materials with lower thermal diffusivities.

While a thermal mass is used in the above described embodiment to facilitate flow of heat away from the surface and distal portion of the return electrode, a heat pipe or circulating fluid can also be used to pull heat away from the body of the return electrode.

The distance between the active and return electrode is also an important factor. If the distance between the electrodes is too small, shorting or arching between the electrodes will occur. If the distance is too large the instrument will be awkward to use and will not be acceptable to the surgeon. Further, the increase distance may increase the overall power requirements. While smaller and/or larger distances are possible, it has been found that having a minimum distance between the two electrodes that falls in the range of 0.1 mm to 3.0 mm works well. The distance between the two electrodes is also limited by the dielectric strength of the insulative material used between the electrodes.

In designing the electrodes it has been found that the difference between the thermal diffusivity of the return electrode and the thermal diffusivity of the active electrode has some effect. Using a material for the active electrode with a thermal diffusivity relatively lower than the thermal diffusivity of the return electrode means the return electrode can be either designed with a material with a lower thermal diffusivity, or, if the return electrode is made of a material with a high thermal diffusivity, the volume of the return electrode can be smaller.

One optimized design that works well uses a volume of high purity silver for the return electrode combined with a tungsten or stainless steel active electrode.

While the above description focuses on using metals with various thermal properties for the electrodes or the electrode surface, electrically conductive materials other than metals, such as a composite, resins, carbon, carbon fiber, graphite, and the like filled composite may also be used for at least one of the electrodes. These materials, or the portion that comes in contact with tissue, need to be biocompatible.

FIG. 4, shows a cross-section view of the electrosurgical instrument tip 114 from FIG. 2 looking along the longitudinal axis LA. The view shows return electrode has a substantial volume as compared to active electrode 116, although the sizes are not drawn to scale. Return electrode also is shown as constructed from a core of material 124 and plated or coated with a surface treatment 124a of high thermal diffusivity material. A core material 124, such as stainless steel, tungsten, nickel or titanium that provides structural stability may be optimal. In some applications, materials such as aluminum or copper may be used as the core and because they have higher thermal diffusivity, the size of the return electrode may be reduced. As discussed previously, a volume of material with a high thermal diffusivity is required in the construction of the return electrode. If a material with high thermal diffusivity, such as silver, is plated or coated over a core material with lower thermal diffusivity, such as nickel, the coating material should have a sufficient thickness to remove heat from the surface of the return electrode and also transport heat away from the proximal portion of the return electrode. When using a stainless steel core and a high purity silver coating, it has been found that a coating of high purity silver of at least 0.002 inches works well. A plating thickness of 0.008 or higher is more desirable. It is anticipated that lower thicknesses can be used for instruments with smaller active electrodes. FIG. 4 shows a circular cross section of the return electrode 118 and the active electrode 116. Cross sections other than circular for either or both electrodes can also be used. As an example, the shape of the cross section of the return electrode 118 can be a narrow ellipse, rectangular, trapezoidal, or random. It is believed an elliptical shape will in fact improve the visibility of the active electrode when the surgeon is cutting and looking down the side of the instrument. Asymmetric cross sections could also be beneficial in some types of surgery.

FIG. 5 shows another electrosurgical instrument tip. Electrosurgical instrument tip 50 is similar to electrosurgical instrument tip 114 explained above. Electrosurgical instrument tip 50 in this embodiment is arranged in a geometry that resembles a traditional electrosurgical blade. Electrosurgical instrument tip 50 includes an active electrode 125 and return electrode 126. Return electrode has an edge 126e extending around a portion of the surface. Active electrode 125 is proximate the edge 126e of return electrode 126. Separating active electrode 125 and return electrode 126 is an insulative material 127, which is normally made of a plastic or ceramic or other dielectric material. The insulative separation between electrodes 125 and 126 may be air or some other gas in some cases. Insulative material should be proximate edge 126e as well. Active electrode 125 may be constructed from a material with a high melting point. Active electrode 125 is shown as extending contiguously around return electrode 126, but active electrode may be non-contiguous as well. The electrosurgical instrument tip 50, or the blade, is held in a connector housing 129 similar to housing 123.

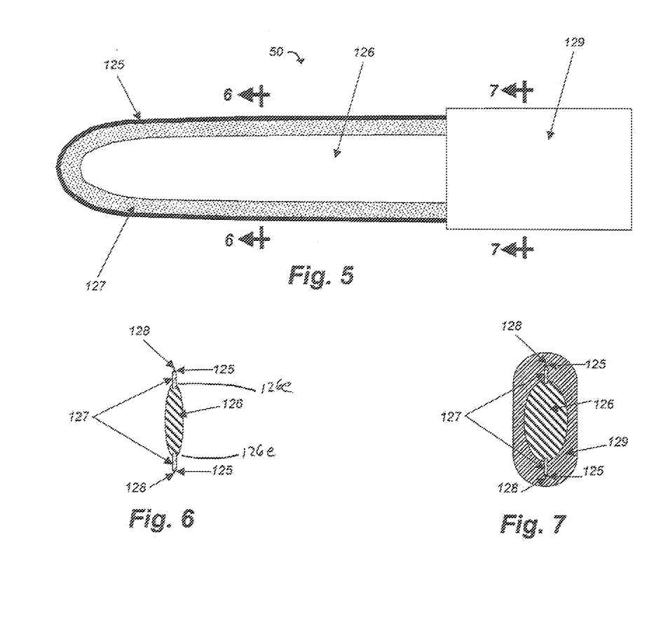

FIGS. 6 and 7 are cross sections of the electrosurgical instrument tip 50. The active electrode 125 may be sharpened to an active electrode edge 128 to facilitate a higher electrical current concentration. The volume of the return electrode 126 is substantial and as the cross sectional area of the return electrode stays the same or increases moving away from the distal tip, heat flow away from the return electrode is facilitated. This prevents return electrode and the blade as a whole from sticking or dragging, a major disadvantage of the prior art.

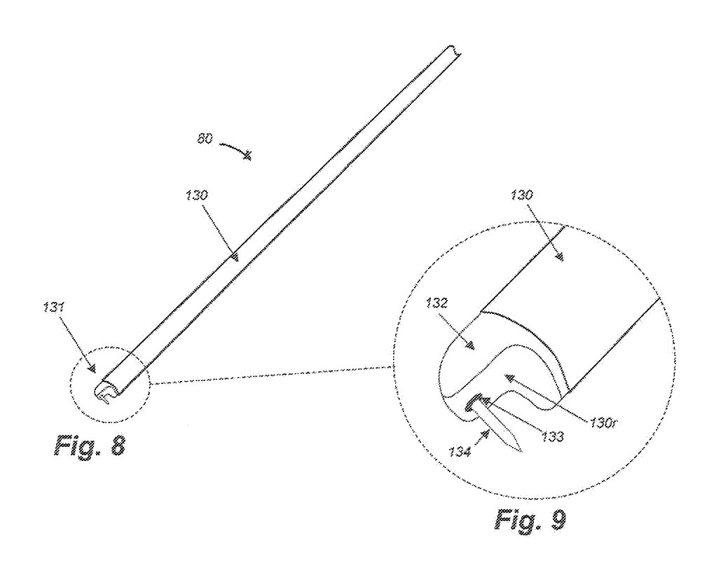

FIG. 8 shows an embodiment of the invention adapted as an endoscopic 80 tool for endoscopic use. Endoscopic tool 80 has a handle or shaft 130. Shaft 130 may be made from an electrically insulative material or wrapped in an electrically insulative sleeve. Tool 80 terminates at a distal tip 131. Tool 80 normally connects or plugs into a handle such as housing 123 or 129, not specifically shown.

FIG. 9 shows a detail of the tip 131 of the tool 80. Tip 131 includes a recess area 130r for the active electrode 134. A return electrode 132 is exposed at tip 131. An active electrode 134 is separated electrically from return electrode 132 by an electrically insulative material 133. In this illustration the active electrode exits the shaft 90 degrees to the axial portion of the electrode, but other angular configurations are possible. This configuration is especially useful for laparoscopic cholecystectomy (endoscopic surgical removal of the gallbladder). Dissipation of heat from the return electrode is facilitated as with previous embodiments with a volume of high thermal diffusivity material (not shown) that extends proximally back into shaft 130. This instrument can also be configured with the active electrode shaped like a blade, spoon, hook, loop or other configuration to better facilitate a range of endoscopic procedures. The active electrode can also exit the instrument axially from the distal tip for the same reason.

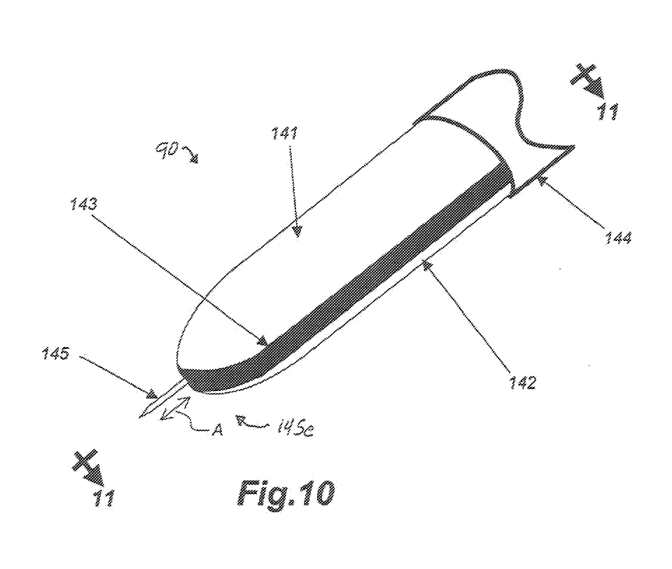

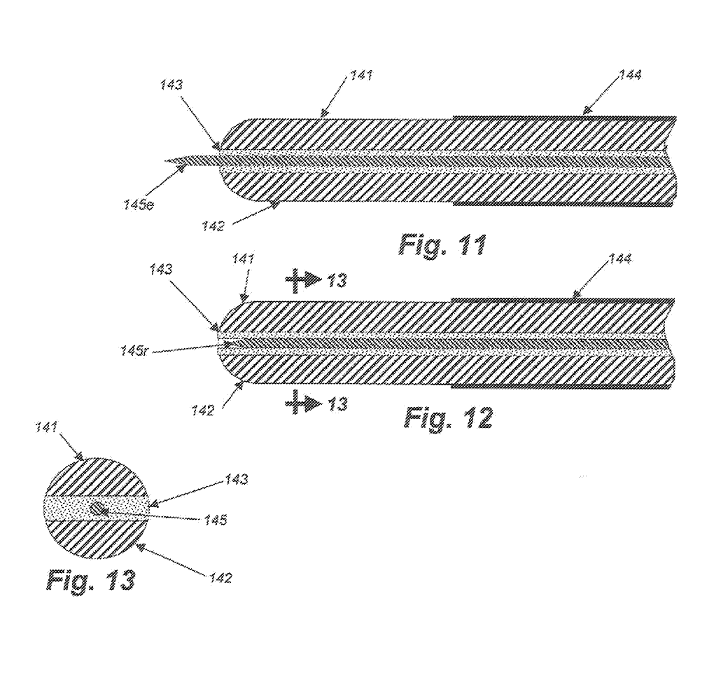

FIG. 10 shows another embodiment of the invention including electrosurgical instrument tip 90. The electrosurgical instrument tip 90 include active electrode 145 and return electrodes 141 and 142. Insulative material 143 separates return electrodes 141 and 142, and active electrode 145. As shown by directional arrow A, active electrode 145 is movable with relation to return electrodes 141 and 142. Thus, active electrode 145 has extended position 145e (as shown in FIGS. 10 and 11) and a retracted position 145r (as shown in FIG. 12).

This embodiment allows the surgeon to cut and coagulate using a single bipolar instrument. Return electrodes 141 and 142 are separated electrically. During use a surgeon can extend active electrode 145 to cut tissues. In the cutting mode, return electrodes 141 and 142 may or may not be coupled. However, during a procedure if the surgeon needs to coagulate, active electrode 145 is retracted. While retracted, electrical power is provided to one of the return electrodes 141 or 142 while the other remains grounded, providing bipolar coagulation action for low power coagulation. As can be appreciated, in the extended position, the electrosurgical instrument tip 90 functions similar to the electrosurgical instrument tip 114 as shown in FIGS. 2 and 3. Different electrosurgical waveforms are normally used for coagulation vs. cutting and these waveforms are well known to those versed in the art of electrosurgery. The mechanism used to extend and retract the active electrode 145 also can be used to signal the generator to switch to the appropriate waveform for cutting when the active electrode is extended or coagulation when the active electrode is retracted. For coagulation this mechanism will also switch the connection of the generator positive and ground to electrodes 141 and 142 respectively. Switching electrical power could be accomplished using actuator 111.

FIG. 13 shows the cross section of the embodiment including the electrically insulative material 143 that separates the two return electrodes 141 and 142 and also contains the active electrode 145 used during cutting. The design of the cauterization electrodes illustrated in this embodiment consists of two electrodes opposed to each other, however, other anticipated configurations include two or more coaxial electrodes, multiple pie shaped electrodes or other electrode geometries.

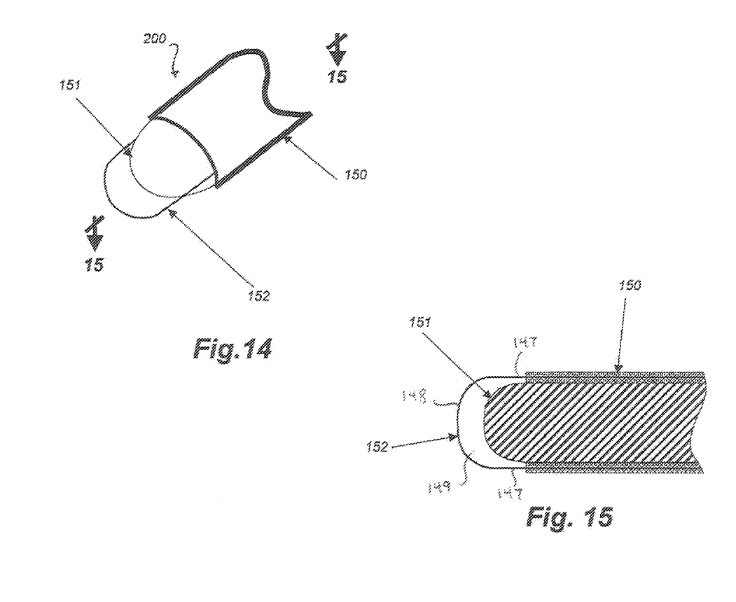

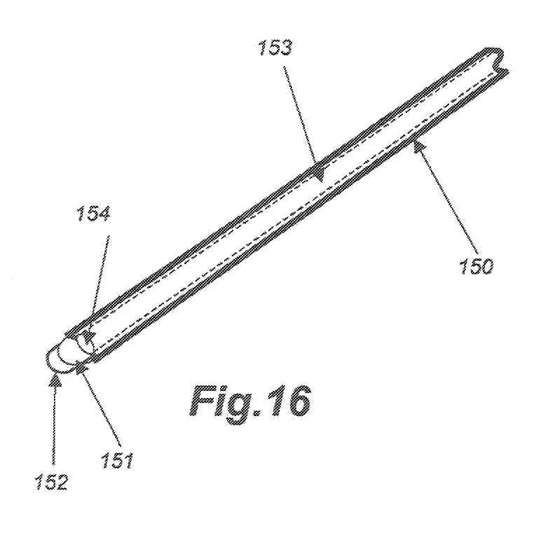

FIG. 14 shows an electrosurgical instrument tip useful for bipolar resection of tissue comprising a return electrode 151 and a loop active electrode 152. Other than the shape, instrument 200 operates similar to those described above. Instrument 200 may be provided with a suction canella 153 as shown in FIG. 16. Suction canella 153 removes tissue and body fluid from the surgical site through the distal end of the canula 154 so the surgeon can continue the procedure. The end of the cannula opposite of the opening 154 (proximal end) is coupled to a suction source (not shown) and a hole in the side of the canula 153 may be incorporated to allow the surgeon to control the suction as is well known in the art. Suction canella 153 could be used with multiple embodiments described. In this embodiment the active electrode 152 is in the shape of a semicircle or loop. The ends of the active or loop electrode are captured within the insulating housing 150. The return electrode 151 in this embodiment is semi-spherical, however could be made in various shapes. As the loop electrode 152 is drawn across the tissue it cuts down, thus facilitating easy and precise removal of larger volumes of tissue.

FIG. 15 is a cross section view of instrument 200 showing the loop active electrode 152, the insulating housing 150 and the return electrode 151. This view shows the ends of the active electrode 152 captured within the insulating housing 150.

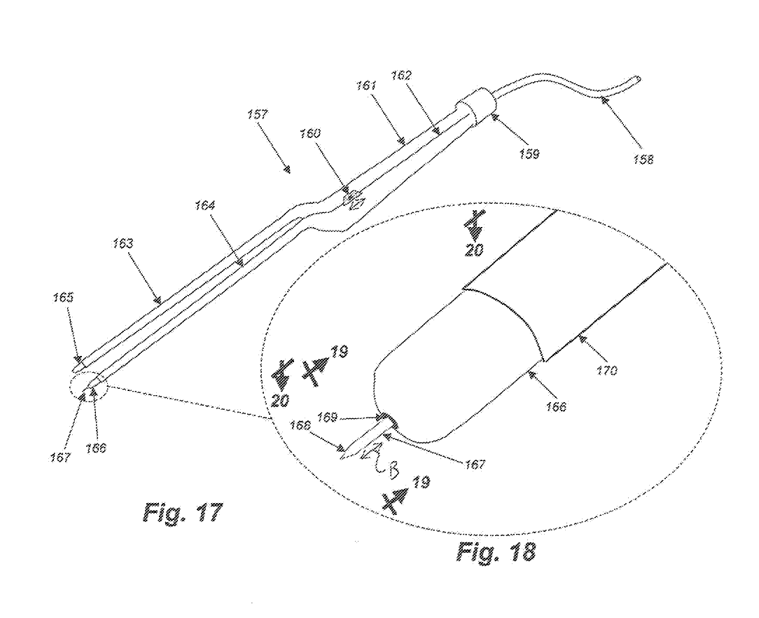

FIGS. 17 through 23 show the present invention incorporated into a bipolar electrosurgical forceps. This instrument allows the surgeon to grasp tissue, coagulate the tissue within the jaws of the bipolar forceps and cut or resect tissue using a single bipolar instrument.

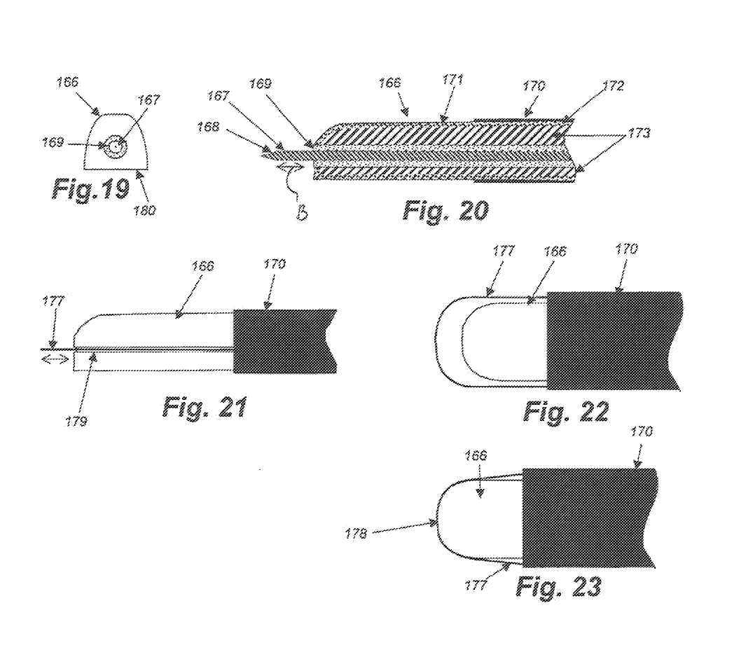

FIG. 17 shows the bipolar forceps 157 with the handles 161 and 162, the tines 163 and 164 and the forceps tips 165 and 166. The bipolar forceps is connected to the generator through a connector 159 and a cable 158 known to those experienced in the art. At least one of the forceps tips is coated with or made of a high thermal diffusivity material as discussed previously. This material prevents the forceps tips from sticking during coagulation. It also allows one or both of the forceps tips 165 and 166 to act as the return electrode per the present invention. A mechanism 160 in the forceps allows the forceps active electrode 167 to be extended or retracted as shown previously in FIG. 10. Mechanism 160 may be a thumb slider as shown that allows the user to extend and retract the active cutting electrode 167 and also switches the waveform and electrical connections as discussed previously. Referring to FIG. 18, the detail of the cutting tip of the forceps is shown. The active electrode 167 can be extended or retracted. It is electrically separated from electrode 166 by an insulative material 169 that runs down the length of the interior of the instrument (not shown), which is similar to the device shown in FIG. 3. The tip of the active electrode may be sharpened to an edge 168 or other shape such as a point, wedge, dowel, blade, hook or the like. The bipolar forceps are normally coated with a layer of insulation 170, normally a plastic such as nylon. This provides an electrical insulation barrier between the instrument and the surgeon. An end view of the tip of the instrument shown in FIG. 18 is shown in FIG. 19. The instrument may be provided with a flat face 180 located on the inside of the forceps to facilitate grasping of tissue. A cross-section view of the tip shown in FIG. 18 is shown in FIG. 20. FIG. 20 shows the insulation 169 that runs down the instrument tine and electrically separates the active cutting electrode 167 from the return electrode 166. The movement of active electrode 167 relative to electrode 166 is represented by arrow B.

While the whole tip of the forceps, or return electrode 166 (sometimes referred to as forceps tip 166) can be made of a high thermal diffusivity material, FIG. 20 shows a return electrode 166, or forceps tip, that is coated with the high thermal diffusivity material. The underlying core 173 of the forceps tip is made of a material to give the forceps structural strength. As discussed previously, appropriate core 173 materials include stainless steel, tungsten, nickel or titanium. The core is then coated or plated with a high thermal diffusivity material 172. When silver of a purity level of over 90% is used an appropriate thickness for the coating or plating of high thermal diffusivity material has been found to be a relatively thick layer of about 0.002 inches or more. Experience has shown that with plating of 0.002 inches thick, the plating should also extend back from the very tip of the forceps by a length of at least 1.0 inches to facilitate dissipation of heat from the tip area. Thicker plating may require less length of plating and plating thicknesses of over 0.008 inches have been used.

FIGS. 21 through 23 show a forceps tip with a loop electrode for dissecting tissue. The loop active cutting electrode 177 can be extended or retracted using the mechanism discussed previously. When retracted the loop wire may nest in a groove 179 in the forceps tip 166. This prevents the loop from getting in the way when using the forceps in coagulation and grasping mode. Return electrode 166 is made of high thermal diffusivity material as discussed previously.

When the surgeon wishes to resect tissue, the loop electrode can be extended as shown on FIG. 22. The loop can then be retracted as shown in FIG. 23 and the bipolar forceps can be used for grasping and coagulation.

An embodiment of the present invention and many of its improvements have been described with a degree of particularity. It should be understood that this description has been made by way of example, and that the invention is defined by the scope of the following claims.

We claim:

1. An electrosurgical instrument, comprising:
a housing formed from an electrically insulating material, said housing having a distal end;
an insulating layer, said insulating layer extending distally from said distal end of said housing and comprising an exposed outer surface and a distal end;
a first electrode and a second electrode, said first and second electrodes positioned on said outer surface of said insulating layer so as to be spaced apart from each others; and
a third electrode having a distal tip, said third electrode movably mounted within said insulting layer so as to move between an extended position and a retracted position in which said distal tip is exposed (positioned outward/distally/forward of said distal end of said insulating layer) when in said extended position and is positioned within said insulating layer when in said retracted position;
wherein when said third electrode is in said extended position, said electrosurgical instrument functions as a cutting instrument, and wherein when said third electrode is in said retracted position, said electrosurgical instrument functions as a coagulation instrument.
